(19) United States
(12) Patent Application Publication
(10) Pub. No.: US 2012/0301522 A1
(43) Pub. Date: Nov. 29, 2012

(54) PEROXIDE-STABILIZED ABRASIVE TOOTH WHITENING COMPOSITIONS, PROCESS FOR PREPARING AND METHOD OF USE

(75) Inventors: William E. Prosise, Ramsey, NJ (US); Robert J. Corring, Rockaway Township, NJ (US); David Vaughan Palmer, Horsham (GB); David Rees, Hamworthy (GB)

(73) Assignee: ISP Investments Inc., Wilmington, DE (US)

(21) Appl. No.: 13/474,297

(22) Filed: May 17, 2012

Related U.S. Application Data
(60) Provisional application No. 61/487,974, filed on May 19, 2011.

(51) Int. Cl. A61K 8/81; A61Q 11/00

(52) U.S. Cl. 424/401; 424/53

(57) ABSTRACT

Disclosed herein is a water-based, peroxide-stabilized, abrasive tooth whitening composition comprising: (i) a peroxide-polymer complex comprising (a) a peroxide component or a peroxide generating component; and (b) a vinyl lactam based polymer; (ii) coated abrasive particles; and one or more orally acceptable carriers. This application also provides methods for whitening the tooth surfaces by contacting with the compositions. Additionally disclosed is a process for preparing the compositions.

### CROSS REFERENCE TO RELATED APPLICATIONS

This application claims the benefit of U.S. Provisional Patent Application Ser. No. 61/487,974, filed May 19, 2011 and entitled PEROXIDE-STABILIZED ABRASIVE TOOTH WHITENING COMPOSITIONS, PROCESS FOR PREPARING AND METHOD OF USE, which is hereby incorporated by reference in its entirety.

### FIELD OF THE INVENTION

The present application relates to oral care compositions and methods, and more particularly, to a water-based, peroxide-stabilized, abrasive tooth whitening composition.

### BACKGROUND OF THE INVENTION

Human teeth normally start out white and shiny due to their smooth enamel surface. Over time, the enamel is gradually worn down from the effects of chewing and acids generated by various foods. This causes the enamel to become thinner and more transparent, allowing the darker color of the internal dentin to become visible. Additionally, the worn enamel will develop fine cracks and pitting that will provide sites where stains can take hold and give teeth a darker, less shiny appearance.

Materials found in commercial toothpaste such as abrasives and condensed phosphates can remove the extrinsic (superficial) stains from teeth but can only whiten teeth to the extent that they will appear as they did before extrinsic staining occurred. To remove intrinsic stains (those in the interior of the tooth caused by normal aging, trauma and absorption) bleaching is the only effective method available. Initially bleaching was done as an in-office procedure by a dentist using hydrogen peroxide. This was followed by the introduction of take-home whitening kits that were used under the direction of a dentist. Both of these options are relatively expensive. More recently, over the counter whitening kits have become available. These are more affordable but not as effective as professionally controlled procedures. Unfortunately, this type of teeth whitening is not a permanent solution. The process of darkening and staining will continue after whitening treatments are discontinued. As such, the most effective way to maintain the white appearance of one's teeth is to apply treatment on a regular basis. The easiest way to do this is through the daily use of toothpaste that provides a bleaching agent and functional materials commonly found in a commercial toothpaste composition for tooth whitening purpose.

The majority of tooth whitening formulae in use currently employ a suitable form of peroxide source as a whitening agent. The effectiveness of peroxide source in oral care has been long recognized. Peroxide has been used by dental clinicians for decades as an oral antiseptic. Such compounds have proven effective in the treatment of gingivitis, oral lesions, periodontitis, and herpetic stomatitis and in combating plaque.

Such tooth whitening compositions having peroxide compounds normally do not contain abrasive polishing agents, because, these abrasive agents facilitate the rapid destabilization of the peroxide compounds, and thereby, the oxygen whitening agent is prematurely released. So, the evolution of gas is undesirable with toothpaste or gel composition as it leads to swelling or bursting of tubes containing such compositions. Further, capped tubes filled with tooth whitening composition having peroxy compounds and silica abrasives tend to explode within a few days after filling. When alumina abrasives are replaced for silica, the filled product is pocketed with gas bubbles within days of filling.

Formulating tooth whitening composition without abrasive agents has less stain removal properties as compared to tooth whitening compositions comprising abrasive agent. The abrasive agent is included in a tooth whitening composition to debride and physically scrub the external surface portion of teeth. This scrubbing act removes biofilms of microbes, plaque layers, stains and discoloring pigments that are found on teeth that cause undesired discoloration. Moreover, these abrasive agent microabrade the tooth in order to give enamel more gloss.

U.S. Pat. No. 5,256,402 discloses abrasive dentifrice compositions containing oxygen liberating whitening compound which is stable with respect to oxygen level and exhibits heightened and rapid whitening of teeth and stain removal which comprises a combination of a dicalcium phosphate compound and a metal ion free peroxide compound.

U.S. Pat. No. 5,814,304 discloses an aqueous abrasive whitening composition containing a peroxide whitening compound which is chemically and physically stable and exhibits heightened and rapid whitening of teeth and stain removal which comprises a combination of water, calcined alumina abrasive and a peroxide compound.

U.S. Pat. No. 5,851,514 discloses an aqueous abrasive whitening composition containing a peroxide whitening compound which is chemically and physically stable and exhibits heightened and rapid whitening of teeth and stain removal which comprises a combination of water, abrasive and peroxide compounds, a humectant containing a polyethylene glycol, a polyoxyethylene-polyoxypropylene block copolymer, a metal ion complexing agent and an antioxidant, the pH of the composition being in the range of about 5.8 to about 7.2.

U.S. Pat. No. 4,788,052 discloses a stable aqueous hydrogen peroxide gel dentifrice for oral anti-gingivitis application having an acid pH of about 3-6 comprising a combination of hydrophilic and hydrophobic fumed silica gelling agent, hydrogen peroxide, about 20-40% by weight of a polyethylene glycol humectant, flavor, sweetening agent, sodium benzoate, and a nonionic surfactant as the essential ingredients, prepared by a process which comprises controlling the order of addition of the essential ingredients. Distilled (deionized) water is preferred in the formulation to prevent minimal contamination.

US Patent Publication No. 20090117058 discloses a toothpaste composition having tooth whitening effect, and more specifically, to a toothpaste composition which contains hydrogen peroxide, as peroxide releasing oxygen free radicals for the whitening effect, and silica with reduced contents of metal ions, to overcome the difficulty in long-term storage due to the release of oxygen free radicals resulting from degradation of peroxide by metal ions released from other components in the composition, and unsatisfactory tooth whitening effect. The toothpaste composition of the present invention does not release oxygen free radicals during storage, and so has excellent preservativeness and sustained tooth whitening effect.

U.S. Pat. No. 5,766,574 discloses a dual component whitening dentifrice composition which comprises a first dentifrice component containing a peroxide compound such as urea peroxide and a second dentifrice component containing an abrasive such as alumina or silica which is incompatible with the peroxide, the first and second dentifrice components being maintained separate from the other until dispensed and combined for application to teeth requiring whitening.

US Patent Publication No. 20060045854 discloses oral care compositions comprising: (a) a peroxide complex comprising a peroxide component and an N-vinyl heterocyclic polymer (e.g., poly-N-vinyl polylactam, or poly-N-vinyl-polyimide); (b) a whitening agent (e.g., hydrogen peroxide); and (c) an orally acceptable carrier. In one embodiment, the carrier comprises a film forming material. Methods are also provided for making an oral care composition comprising: (a) mixing a whitening agent, silicone adhesive and carrier fluid to form a homogenous mixture; (b) adding a peroxide complex to said homogenous mixture, wherein said complex comprises hydrogen peroxide and an N-vinyl heterocyclic polymer; and (c) mixing under vacuum.

PCT Publication No. 2007037961 describes a single phase whitening dentifrice that includes (i) a whitening agent selected from the group consisting of hydrogen peroxide, a bound peroxide and a solid peroxide (ii) an abrasive and (iii) a substantially anhydrous orally acceptable carrier, for example, polyethylene glycol. The bound peroxide may be hydrogen peroxide and a polymer and/or any peroxide compound and a porous cross-linked polymer, such as polymers of polyvinyl pyrrolidone, polyacrylates, a polymethacrylates, and a polyitaconates. The solid peroxide may be sodium perborate or urea peroxide. The invention also provides methods of whitening the tooth surfaces by contacting the surface with the composition.

In view of foregoing, it is an objective of the present application to provide a water-based tooth whitening composition comprising both abrasive agent and peroxide source in a single phase, wherein, the peroxide source is significantly stabilized by completely disabling the contact between abrasive agent and peroxide source by providing appropriately coated abrasive agents. Such coated abrasive agent substantially physically prevents peroxides from interacting with stability-limiting abrasives.

### SUMMARY OF THE INVENTION

What is described herein is a water-based, peroxide-stabilized, abrasive tooth whitening composition comprising: (i) a peroxide-polymer complex comprising (a) a peroxide component or a peroxide generating component and (b) a vinyl lactam based polymer; (ii) coated abrasive particles; and (iii) one or more orally acceptable carriers.

According to one important aspect of the present application, there is provided a water-based toothpaste composition comprising (i) coated abrasive particles and (ii) an incompatible peroxide component source.

According to another aspect of the application, there is provided a method for whitening teeth comprising preparing an abrasive tooth whitening composition and applying on to the teeth by means of flexible trays, forms, toothbrush, freehand or fingers.

It has been discovered the tooth whitening composition of the present application is beneficial over the earlier known compositions comprising abrasive and peroxide source. The present water-based abrasive tooth whitening composition is able to demonstrate superior tooth whitening property because of significantly enhanced stabilized peroxide component.

### DESCRIPTION OF DRAWINGS AND FIGURES

FIG. 1—Stability of peroxide content (Peroxidone K-30) in an aqueous system comprising encapsulated abrasive vs. unencapsulated abrasive conducted at 40° C.

FIG. 2—Stability of peroxide content (Peroxidone K-30) in an aqueous system comprising encapsulated abrasive vs. unencapsulated abrasive conducted at Room Temperature (RT).

FIG. 3—Abrasivity test conducted for encapsulated abrasive vs. unencapsulated abrasive after 2, 5 and 10 brushes.

### DETAILED DESCRIPTION OF THE INVENTION

While this specification concludes with claims particularly pointing out and distinctly claiming that which is regarded as the invention, it is anticipated that the invention can be more readily understood through reading the following detailed description of the invention and study of the included examples.

By the term “comprising” herein is meant that various optional, compatible components can be used in the compositions herein, provided that the important ingredients are present in the suitable form and concentrations. The term “comprising” thus encompasses and includes the more restrictive terms “consisting of” and “consisting essentially of” which can be used to characterize the essential ingredients, peroxide-polymer complex, coated abrasives, water-soluble surfactants, and additives if any.

All percentages, parts, proportions and ratios as used herein, are by weight of the total composition, unless otherwise specified. All such weights as they pertain to listed ingredients are based on the active level and, therefore; do not typically include solvents or by-products that may be included in commercially available materials, unless otherwise specified.

All references to singular characteristics or limitations of the present invention shall include the corresponding plural characteristic or limitation, and vice-versa, unless otherwise specified or clearly implied to the contrary by the context in which the reference is made.

Numerical ranges as used herein are intended to include every number and subset of numbers contained within that range, whether specifically disclosed or not. Further, these numerical ranges should be construed as providing support for a claim directed to any number or subset of numbers in that range.

The term “about” can indicate a variation of 10 percent of the value specified; for example about 50 percent carries a variation from 45 to 55 percent. For integer ranges, the term about can include one or two integers greater than and less than a recited integer.

The patents and publications referred to herein are hereby incorporated by reference to the extent necessary to understand the present invention.

As used herein, the words “preferred,” “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

References herein to “one embodiment,” “one aspect” or “one version” or “one objective” of the invention include one or more such embodiment, aspect, version or objective, unless the context clearly dictates otherwise.

As used herein, the term “polymer” is meant to encompass oligomer, and includes, without limitation, homopolymers, copolymers, terpolymers, etc. The polymers described herein can also be linear, branched and/or crosslinked polymers.

Disclosed herein is a water-based, peroxide-stabilized, abrasive tooth whitening composition comprising: (i) a peroxide-polymer complex comprising (a) a peroxide component or a peroxide generating component and (b) a vinyl lactam based polymer; (ii) coated abrasive particles; and (iii) one or more orally acceptable carriers.

The water-based tooth whitening composition of the present application comprises a water content of from about 30% wt. to about 80% wt. of the composition. The preferred water content is from about 40% wt. to about 70% wt, the most preferred range of water content is from about 55% wt. to about 65% wt. of the composition, wherein, the percentage limits of the water content would include any free water and all water contained in any ingredients.

The peroxide component or peroxide compound of the present application is an oxidizing agent which is capable of oxidizing, and also includes precursors of compounds capable of oxidizing. The oxidizing agent can be any oxidizing agents that are known in the prior-art. In one embodiment, the oxidizing agent for the present application is selected from the following non-limiting group of compounds including peroxides, hydroperoxides, peroxides of alkali and alkaline earth metals, organic peroxy compounds, peroxy acids and their salts. However, the preferred peroxide component or peroxide compound of the present application is selected from the group including but are not limited to hydrogen peroxide, lithium peroxide, potassium peroxide, strontium peroxide, zinc peroxide, sodium peroxide, magnesium peroxide, calcium peroxide, barium peroxide, carbamide peroxide, glyceryl hydrogen peroxide, alkyl hydrogen peroxides, dialkyl peroxides, alkyl peroxy acids, peroxy esters, diacyl peroxides, benzoyl peroxide, monoperoxyphthalate, alkyl peroxy acids, monoperoxyphthalate, percarbonate salts of alkali and alkaline earth metals such as lithium, potassium, sodium, magnesium, calcium and barium, perborate salts of alkali and alkaline earth metals such as lithium, potassium, sodium, magnesium, calcium and barium. The most preferred peroxide component of the present application is hydrogen peroxide. The peroxide component comprises from about 0.1% to about 60%, preferably from about 1% to about 30%, most preferably from about 3% to about 20% of the abrasive tooth whitening composition.

The peroxide-polymer complex of the present application comprises N-vinyl lactam based polymers which are either straight-chained or crosslinked in nature and are capable of absorbing, adsorbing, or complexing with the provided peroxide component of the present application. Also, the polymer is suitably facilitated to retain the peroxide component or peroxide compound of the present application. Such retained peroxide source discharges the peroxide component when it is applied onto the teeth for whitening.

According to one important embodiment of the present application, the vinyl lactam based component of polymers or co-polymers for preparing peroxide-polymer complex is selected from the group consisting of N-vinyl-2-pyrrolidone, N-vinyl-2-piperidone, N-vinyl-3-methyl-pyrrolidinone, N-vinyl-3-methyl-piperidone, N-vinyl-3-methyl-caprolactam, N-vinyl-4-methyl-pyrrolidinone, N-vinyl-4-methyl-2-pyrrolidone, N-vinyl-4-methyl-piperidone, N-vinyl-4-methyl-caprolactam, N-vinyl-5-methyl-pyrrolidinone, N-vinyl-5-ethyl-2-pyrrolidone, N-vinyl-4-methyl-piperidone, N-vinyl-3-ethyl-pyrrolidinone, N-vinyl-4,5-dimethyl-pyrrolidinone, N-vinyl-5,5-dimethyl-pyrrolidinone, N-vinyl-3,3,5-trimethyl-pyrrolidinone, N-vinyl-5-methyl-5-ethyl-pyrrolidinone, N-vinyl-3,4,5-trimethyl-3-ethyl-pyrrolidinone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl-2-caprolactam, N-vinyl-7-methyl-caprolactam, N-vinyl-7-ethyl-caprolactam, N-vinyl-3,5-dimethyl-caprolactam, N-vinyl-4,6-dimethyl-caprolactam, N-vinyl-3,5,7-trimethyl-caprolactam, N-vinyl-2-valerolactam, N-vinyl-hexahydro-2-azepinone, N-vinyl-octahydro-2-azocinone, N-vinyl octahydro-2-azoninone, N-vinyl decahydro-2-azecinone, polyvinylpyrrolidone (PVP), poly-N-vinyl-poly-2-pyrrolidone (PVPP), poly-N-vinypoly-2-piperidone, poly-N-vinyl-poly-2-caprolactam. The monomers for preparing vinyl lactam based polymer of the present application is selected from any monomer which is having 3 to 8 atoms in a heterocyclic ring, comprising a carbonyl carbon atom and an heteroatom (such as N, S, O) in its vinyl moiety. Further, it is understood that embodiments of the present application are not limited to a polymers of a specific molecular weight, any acceptable purity, preferably pharmaceutical grade, is within the scope of this present application.

In a preferred embodiment, soluble and insoluble polymers of the present application would include but are not limited to poly-vinyl-pyrrolidone (PVP), cross-linked PVP (cPVP), povidone, polyvidone, polyvidonum, poly-(N-vinyl-2-pyrrolidinone), poly-(N-vinylbutyrolactam), poly(1-vinyl-2-pyrrolidone), poly-[1-(2-oxo-1-pyrrolidinyl)ethylene, polyvinylpolypyrrolidone, cross-povidone, PVP/VA, alkylated PVP, and any polymer containing VP monomers. The exemplary list of commercial available polymers of the present application would include but are not limited to Peroxydone K30, Peroxydone K90, Peroxydone XL10, Plasdone S-630, Poly-Pore 337HP, PolyPlasdone INF-10, Kollidon, Luvicross, PVP K-Series, Povidone K-30, PVP K-30, PVP VA-64, PVP K-17, PVP K-90.

The tooth whitening compositions comprising incompatible peroxide source and uncoated abrasive particles tend to destabilize the peroxide source of the compositions which results in significantly lesser tooth whitening efficacy. Hence, encapsulating abrasives particles in a suitable coating or encapsulating agent has resulted in considerably more stable tooth whitening compositions. The term encapsulated or coated means that a barrier is provided around the abrasive particles.

Abrasive particles for the present water-based abrasive tooth whitening composition are selected from the group consisting of sodium metaphosphate, potassium metaphosphate, tricalcium phosphate, calcium phosphate dehydrate, anhydrous dicalcium phosphate, calcium pyrophosphate, magnesium orthophosphate, trimagnesium phosphate, calcium carbonate, baking soda, sodium hexametaphosphate, magnesium carbonate, magnesium silicate, titanium dioxide, zinc oxide, aluminum silicate, zirconium silicate, hydrated alumina, bentonite, hydrated silica, amorphous silica, silica gel or colloidal silica, alkali metal aluminosilicate complexes, silicon dioxide, alumina, and any combination thereof. The preferred range of abrasives for the present application is from about 10.0% by wt. to about 40% by wt. of the total composition. The most preferred range is from about 20.0% by wt. to about 30.0% by wt. of the composition. The non-limiting commercially available abrasive agents of the present application are Tixosil 73, Zeodent 113, and Sident 8.

According to the present application, the coated abrasive particles is prepared by employing suitable coating or encapsulating agent selected from the group consisting of algin, alginic acid, alginate salts, camitine, carrageenan, dextrin, karaya gum, agar gum, guar gum, gellan gum, irish moss, vee gum, tara gum, okra gum, gum arabic, acacia gum, amylopectin, pectina or pectin, ghatti gum, natto gum, tragacanth gum, xanthan gum, sclerotium gum, kelp, locust bean gum, psyllium seed, tamarind gum, destria gum, chitosan, starch, modified starch, gelatin, fish gelatin, alkyl and hydroxycellulose, microcrystalline cellulose, hydroxyethylcellulose, poly(acrylic acid), polyethylene glycol, polyvinyl alcohol, polyvinylpyrrolidone, zein, polyols, esters thereof, salts thereof and mixtures thereof. The most preferred encapsulating agent for the present application is fish gelatin and/or gum acacia. The required range of encapsulating agent to encapsulate the desired abrasive agent is from about 5% wt. to about 20% wt, preferably 8% wt. to about 12.0% wt. of the selected abrasive agent.

The coating or encapsulation of abrasives of the present application is carried out by any suitable methods that are known in the prior art, however, the preferred method of encapsulation is complex coacervation.

The present application provides tooth whitening compositions comprising an orally acceptable carrier. As used herein, an “orally acceptable carrier” refers to a substance or combination of substances that are safe for use in the compositions of the present application, wherein, the orally acceptable carrier does not significantly reduce the efficacy of the peroxide-polymer complex. Selection of specific carrier substance is depends on the desired product form, including dentifrices, rinses, gels, paints and etc.

According to the present application, the orally acceptable carrier for the abrasive tooth whitening composition is selected from the group including, but not limited to, anticalculus agents, oral cleaning agents, bleaching or whitening agents, desensitizing agents, dental re-mineralization agents, surfactants, detergents, antibacterial agents, anticaries agents, breath freshening agents, buffering agents, pH modifying agents, sweeteners, cooling agents, humectants, bioadhesive agents, coloring agents, thickening agents, film-forming agents, oral hygiene promoting agents, herbal extracts, essential oils, wetting agents, preservatives, foaming agents, flavoring agents, medicinal ingredients, vitamins, taste masking agents, and solubilizers alone or combinations thereof.

The preferred fluoride source for the present tooth whitening composition may be selected from the group including, but not limited to, inorganic fluoride salts, e.g., soluble alkali metal, alkaline earth metal salts such as sodium fluoride, potassium fluoride, ammonium fluoride, calcium fluoride, copper fluoride, cuprous fluoride, zinc fluoride, barium fluoride, sodium fluorosilicate, tin fluoride, stannous fluoride, ammonium fluorosilicate, sodium fluorozirconate, ammonium fluorozirconate, sodium monofluorophosphate, aluminum mono and/or di-fluorophosphate, fluorinated sodium calcium pyrophosphate, alone or in combination. The preferred range of fluoride content of the present application is from about 0.01% wt. to about 3.0% wt. preferably, 0.03% wt. to about 1.0% wt. of the total composition.

The sweetening agent of the present application may be selected from a wide range of agents, including natural, artificial, water-soluble sweeteners, water-soluble artificial sweeteners, and/or dipeptide based sweeteners. The representative illustrations encompass water-soluble sweetener such as monosaccharides, disaccharides, and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol, hydrogenated glucose syrup and mixtures thereof; and water-soluble artificial sweeteners such as the soluble saccharin salts, i.e., sodium or calcium saccharin salts, cyclamate salts, such as the sodium salt and the like, and the free acid form of saccharin; dipeptide based sweetening agents such as L-aspartyl-L-phenyl-alanine methyl ester and materials; dihydrochalcone; glycyrrhizin; Stevia rebaudiana (Stevioside); and the synthetic sweetener 3,6-dihydro-6-methyl-1,2,3-oxathiazin-4-one-2,2-dioxide, particularly the potassium (Acesulfame-K), sodium and calcium salts thereof. The amount of the sweetener will vary with the type of sweetener selected and the desired level of sweetness. Water-soluble sweeteners derived from naturally occurring water-soluble sweeteners, such as a chlorinated derivative of sucrose is known under the product description of sucralose as well as protein based sweeteners such as thaumatococus danielli (Thaumatin I and II) can be used. Sweetening agents are typically used in tooth whitening compositions at levels of from about 0.05% to about 2%, preferably about 0.1% to about 0.5% by weight of the composition.

Another constituent of the present oral care tooth whitening composition described herein is a humectant, and it serves to keep the compositions from hardening upon contact to air, and certain humectants can also contribute desirable sweetness of flavor to the compositions. Such agents include glycerin, sorbitol, polyethylene glycol, propylene glycol, other edible polyhydric alcohols, poloxamers, and combinations thereof, polyols such as propylene glycol, dipropylene glycol, and hexylene glycol, cellosolves such as methyl cellosolve and ethyl cellosolve, vegetable oils and waxes containing at least about 12 carbons in a straight chain such as olive oil, castor oil and petrolatum and esters such as amyl acetate, ethyl acetate and benzyl benzoate. As used herein “propylene glycol” includes 1,2-propylene glycol and 1,3-propylene glycol. The total amount of humectant generally comprises from about 25% to 50%, preferably from about 30% to 45% by weight of the compositions herein.

In preparing abrasive tooth whitening composition, it is essential to add some thickening agents to provide a desirable consistency. Examples of useful thickening agents include carboxyvinyl polymers, carrageenan, hydroxyethyl cellulose, laponite and water soluble salts of cellulose ethers such as sodium carboxymethylcellulose and sodium carboxymethyl hydroxyethyl cellulose, copolymers of lactide and glycolide monomers, carbomers. Natural gums such as gum karaya, xanthan gum, gum arabic, and gum tragacanth can also be used. Colloidal magnesium aluminum silicate or finely divided silica can be used as part of the thickening agent to further improve texture. Suitable thickening agents include polymeric polyether compounds, e.g., polyethylene or polypropylene oxide (M.W. 300 to 1,000,000), capped with alkyl or acyl groups containing 1 to about 18 carbon atoms. Carbomers are commercially available from B.F. Goodrich as the Carbopol Series. Particularly preferred carbopols include Carbopol 934, 940, 941, 956, and mixtures thereof. Thickening agents are usually present in an amount from about 0.1% to about 25% by weight of the total tooth whitening composition. However, the preferred amount of thickening agent for the tooth whitening composition is in the range of about 0.5% to about 15% by weight, most preferably about 2.0%-10.0% by weight of the total composition.

The preferred thickening of the present application is fumed silica; it is also called pyrogenic silica and refers to amorphous silica formed in the vapor phase. Fumed silica may contain, for example, a few hundred primary particles fused into branched-chain, three-dimensional aggregates. Examples of fumed silica include products available under the trade names AEROSIL OX-50, AEROSIL-130, AEROSIL-150, AEROSIL-200 and/or CAB-O-SIL.

The suitable surfactant for preparing tooth whitening compositions may be selected from anionic, non-ionic, amphoteric, cationic and mixtures thereof. The following types of surfactants are representative of the surfactants that can be used:

(a) Anionic Surfactants: Anionic surfactants are particularly useful in accordance with certain embodiments of the present application. Surfactants of the anionic type that may be useful include: Sulfonates and Sulfates: Suitable anionic surfactants include sulfonates and sulfates such as alkyl sulfates, alkylether sulfates, alkyl sulfonates, alkylether sulfonates, alkylbenzene sulfonates, alkylbenzene ether sulfates, alkylsulfoacetates, secondary alkane sulfonates, secondary alkylsulfates, alkyl sulfosuccinates and the like. Further, examples of anionic surfactants include water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Phosphates and Phosponates: Suitable anionic surfactants also include phosphates such as alkyl phosphates, alkylether phosphates, aralkylphosphates, and aralkylether phosphates. Examples include a mixture of mono-, di- and tri-(alkyltetraglycolether)-o-phosphoric acid esters generally referred to as trilaureth-4-phosphate.

(b) Amphoteric Surfactants: Surfactants of the amphoteric type include surfactants having tertiary amine groups which may be protonated as well as quaternary amine containing zwitterionic surfactants. Those that may be useful include: Ammonium Carboxylate Amphoterics: Examples of such amphoteric surfactants include, but are not limited to: certain betaines such as cocobetaine and cocamidopropyl betaine (commercially available under the trade designations MACKAM CB-35 and MACKAM L from McIntyre Group Ltd., University Park, III); monoacetates such as sodium lauroamphoacetate; diacetates such as disodium lauroamphoacetate; amino- and alkylamino-propionates such as lauraminopropionic acid (commercially available under the trade designations MACKAM IL, MACKAM 2L, and MACKAM 151L, respectively, from Mclntyre Group Ltd.). Ammonium Sulfonate Amphoterics: These classes of amphoteric surfactants are often referred to as “sultaines” or “sulfobetaines”. Examples include cocamidopropylhydroxysultaine (commercially available as MACKAM 50-SB from Mclntyre Group Ltd.).

(c) Nonionic Surfactants: Surfactants of the nonionic type that may be particularly useful include: Polyethylene oxide extended sorbitan monoalkylates (i.e., Polysorbates); Polyalkoxylated alkanols: Surfactants such as those commercially available under the trade designation BRIJ from ICI Specialty Chemicals, Wilmington, Del. having an HLB of at least about 14 may be useful; Polyalkoxylated alkylphenols: Examples of surfactants of this type include polyethoxylated octyl or nonyl phenols having HLB values of at least about 14, which are commercially available under the trade designations ICONOL and TRITON. Sulfated and phosphated derivatives of these surfactants may also be useful. Examples of such derivatives include ammonium nonoxynol-4-sulfate, which is commercially available under the trade designation RHODAPEX CO-436; Polaxamers: Surfactants based on block copolymers of ethylene oxide (EO) and propylene oxide (PO) may also be effective. Both EO-PO-EO blocks and PO-EO-PO blocks are expected to work well as long as the HLB is at least about 14, and preferably at least about 16. Such surfactants are commercially available under the trade designations PLURONIC and TETRONIC; Polyalkoxylated esters: Polyalkoxylated glycols such as ethylene glycol, propylene glycol, glycerol, and the like may be partially or completely esterified, i.e., one or more alcohols may be esterified, with a (C₈ to C₂₂) alkyl carboxylic acid. Such polyethoxylated esters having an HLB of at least about 14, and preferably at least about 16, may be suitable for use in compositions of the present invention; Alkyl Polyglucosides: Alkyl polyglucosides may also be used. Examples include glucopon 425, which has a (C₈ to C₁₆) alkyl chain length with an average chain length of 10.3 carbons and 14 glucose units.

(d) Cationic Surfactants: Surfactants of the cationic type that may be useful include but are not limited to primary amines, secondary amines, tertiary amines, quaternary amines, alkanolamines, mono-alkyl alkanolamines, di-alkyl alkanolamines, tri-alkyl alkanolamines, alkyl mono alkanolamines, alkyl di-alkanolamines, alkylamines, mono-alkyl amines, di-alkyl amines, tri-alkylamines, alkoxylated amines, alkyl and aryl amine alkoxylates, methoxylated alkylamines, ethoxylated alkylamines, alkoxylated alkanolamines, alkyl alkanolamines, alkoxylated ethylene diamine derivatives, alkyl/aryl/arylalkyl amine oxides. The preferred cationic surfactants of the present invention would include, but are not limited to, (a) alkyl alkanolamines; and (b) alkyl tertiary amines. Additional information on useful cationic surfactants for the purpose of present invention is well described in McCutcheon's Detergents and Emulsifiers, North American Ed., 1982 and Kirk-Othmer, Encyclopedia of Chemical Technology, 3^{rd} Ed., Vol. 22, pp. 346-387, the contents of which are included herein by reference. The preferred amount of surfactant for the tooth whitening composition is the range of about 0.5% to about 5% by weight, most preferably about 1.0%-2.0% by weight of the total composition.

The coloring agents can be used to create the preferred color for the tooth whitening composition. The coloring agents or colorants used in the present application include natural food colors and dyes suitable for food, drug and cosmetic applications. These colorants are also known as F.D. & C. dyes and lakes and are preferably water-soluble in nature. Examples of representative colorants include, but are not limited to, disodium salt of 5,5-indigotindisulfonic acid (Blue No. 2), 4-[4-(N-ethyl-p-sulfoniumbenzylamino)diphenylmethylene]-[1-(N-ethyl-N-p-sulfoniumbenzyl)delta-2,5-cyclohexadien eimine] (Green No. 1), Yellow No. 10, Green No. 3 comprising a triphenylmethane dye, FD&C Blue #1, FD&C Yellow #5, FD&C Yellow #10, FD&C Red #3, FD&C Red #40; caramel color or powder (#05439), chocolate shade (#05349), green lake blend (#09236), kowet titanium dioxide (#03970), yellow liquid color (#00403), and nitrites. A full recitation of all F.D. & C. and D. & C. dyes and their corresponding chemical structures may be found in the Kirk-Othmer Encyclopedia of Chemical Technology, Volume 5, pages 857-884, which text is accordingly incorporated herein by reference. These coloring agents may be incorporated in amount up to about 1%, more particularly up to about 0.5%, and in some cases less than about 0.1% by weight of the oral care tooth whitening composition.

The flavoring agents useful for this application are any food grade or pharmaceutically acceptable one. Preferably, the flavoring agent comprises natural flavoring oils selected from the group including, but not limited to, essential oils such as peppermint oil, spearmint oil, other mint oils, clove oil, wintergreen oil, anise oil, tea tree oil, lavender oil, pine oil, lemongrass oil, lemon oil, parsley oil, orange oil, clove oil, thyme oil, grapefruit oil, clove bud oil, aniseed oil, basil oil, black pepper oil, camphor oil, cananga oil, cardamom oil, cassia oil, cedarwood oil, cinnamon bark oil, cinnamon leaf oil, citrus oil, mint-type oils, citronella oil, eucalyptus oil, fennel oil, geranium oil, ginger oil, guaiacwood oil, juniper berry oil, lime oil distilled, litsea cubeba oil, patchouli oil, berry oil, pimento leaf oil, sandalwood oil, sage oil, sassafras oil, vetyver oil, bergamot oils, spice oils, origanum oil, pimento oil, buchu oil, caraway oil, carrot seed oil, copaiba oil, geranium oil, rosemary oil, targette oil, mace oil, nutmeg oil, cypress oil, cinnamon oil, coconut oil, fish oil, palm oil, mineral oil, apricot oil, cassia oil, castor oil, coriander oil, corn oil, cottonseed oil, peanut oil, soyabean oil, vegetable oil, pine seed oil, abyssinica oil, macadamia nut oil, limnanthes alba oil alone or in combination.

The flavoring agents may comprise compounds selected from the group consisting of menthone, menthyl acetate, neomenthol, piperitone, pulegone, betacaryophyllene, betacaryophyllene-epoxide, alpha-pinene, beta-pinene, germacrene-D, 1,8-cineol, linalool, menthofurane, camphene, beta-hexenyl phenylacetate, d-limonene, 1-limonene, dl-limonene, alpha-citral, beta-citral (geranol), alpha-terpinene, gamma-terpinene, 2-dodecanal, 2-pentenal, cadiene, decylaldehyde, linalool, terpineol, linalyl esters, terpinyl acetate, decanal, C₈ to C₁₀₋₁₂ aldehydes, acids, amyl salicylate, cavacrol, dihydroeugenol, eugenol, hexyl eugenol, hexyl salicylate, isoeugenol, methyl eugenol, methyl isoeugenol, methyl salicylate, tert-butyl cresol, thymol, vanillin, cedrene, cineole, citral, citronellal, citronellol, cymene, paradihydrolinalool, dihydromyrcenol (DH myrcenol), farnesol, hexyl cinnamaldehyde, hydroxycitronallol, hydroxycitronellal, isocitral, linalool, longifolene, menthol, nerol, nerolidiol, phellendrene, terpinene, tetrahydromyrcenol (TH myrcenol), carvacrol, dihydroguaiaretic acid, nerolidole, gamma-decalactone and delta-decalactone, monocaprin, monolaurin, cinnamic acid, decanoic acid, 3-hydroxydecanoic acid, 9-decenoic acid, senecionic acid, nonanol, decanol, nonanal, decanal, amyl propionate, anethole, anisic aldehyde, cis-3-hexenol, damascone, ethyl acetoacetate, isoamyl acetate, menthol laevo, methyl cinnamate, cyclamen aldehyde, diphenyl oxide, ethyl vanilin, eucalyptol, L-methyl acetate, longifolene, menthol crystals, methyl cedryl ketone, methyl chavicol, methyl salicylate, musk ambrette, musk ketone, musk xylol, phenyl ethyl alcohol, vanilin, I-carvone, terpenes, alpha-citronellol, citronellyl acetate, citronellyl nitrile, para-cymene, dihydroanethole, dihydrocarveol, d-dihydrocarvone, dihydrolinalool, dihydromyrcene, dihydromyrcenol, dihydromyrcenyl acetate, dihydroterpineol, dimethyloctanal, dimethyloctanol, dimethyloctanyl acetate, estragole, ethyl-2 methylbutyrate, fenchol, geraniol, geranyl acetate, geranyl nitrile, hexenal, trans-2-hexenol, cis-3-hexenyl isovalerate, cis-3-hexanyl-2-methylbutyrate, hexyl isovalerate, hexyl-2-methylbutyrate, hydroxycitronellal, lonone, isobornyl methylether, linalool oxide, linalyl acetate, menthane hydroperoxide, 1-methyl acetate, methyl hexyl ether, methyl-2-methylbutyrate, 2-methylbutyl isovalerate, myrcene, nerol, neryl acetate, 3-octyl acetate, phenyl-ethyl-2-methylbutyrate, cis-pinane, pinane hydroperoxide, pinanol, pine ester, alpha-pinene oxide, plinol, plinyl acetate, pseudo lonone, rhodinol, rhodinyl acetate, alpha-terpinene, gamma-terpinene, terpinene-4-ol, terpinolene, terpinyl acetate, tetrahydrolinalool, tetrahydrolinalyl acetate, phellandrene, pinene, methylheptenone, safrol, eugenyl acetate, caryophyllene, borneol, bornyl esters, camphor, menthyl esters, safrole, acetaldehyde, chavicol, cinnamyl acetate, α-thujone, β-thujone, fenchone, naturally available disinfecting plant products/extracts like neem, turmeric, cloves, alone or in combination.

The pH of the composition is generally in the range of about 4 to about 9 or 10. The pH can be controlled with acid (e.g., citric acid or benzoic acid) or base (e.g., sodium hydroxide) or buffered (as with sodium citrate, benzoate, carbonate, or bicarbonate, disodium hydrogen phosphate, sodium dihydrogen phosphate, etc.).

The present compositions may optionally include active agents, such as antimicrobial/anti-plaque agents, dentinal desensitizing agents, etc. An anticalculus agent such as sodium tripolyphosphate, tetrapotassium or tetrasodium pyrophosphates, or mixtures thereof, can also be present in the composition, typically in concentrations from about 0.5% to about 8% by weight.

Examples of antimicrobial agents include, but are not limited to, zinc salts, stannous salts, cetyl pyridinium chloride, chlorhexidine, triclosan, triclosan monophosphate, and flavor oils. Examples of dentinal desensitizing agents to control hypersensitivity include, but are not limited to, salts of potassium, calcium, strontium and tin including nitrate, chloride, fluoride, phosphates, pyrophosphate, polyphosphate, citrate, oxalate and sulfate. Another method for controlling hypersensitivity involves the use of a mechanical shield around the nerve by blocking of the dentinal tubules wholly or partially with tubule blocking agents. Examples of tubule blocking agents include, but are not limited to, cationic alumina, clays, water-soluble or water-swellable polyelectrolytes, maleic acid copolymers and polyethylene particles.

A preservative or preservative system may be employed to prepare tooth whitening compositions comprising beeswax, d-limonene, monohydric alcohol, L-ergothioneine, ascorbic acid, sodium nitrates, sodium erythorbate, erythorbic acid, sodium succinate, grape seed extract, pine bark extract, apple extract, tea proplyphenols, succinic acid, parabens, sodium dehydro acetate, allicin, isothiocyanates, sodium benzoate, potassium sorbate, parahydroxy benzoic acid and salts thereof, (C₁-C₆) alkyl parahydroxy benzoates, sorbic acid and salts thereof, ortho hydroxybenzoic acid and salts thereof, benzoic acid and salts thereof, (C₁-C₄) alkyl benzoates, propionic acid and salts thereof, dehydroacetic acid and salts thereof, formic acid and salts thereof, and undec-10-enoic acid or salts thereof alone or in combination. Preservatives or preservative systems including sodium benzoate, methyl-4-hydroxybenzoate, phenylcarbinol, benzalkonium chloride and/or thimerosal are particularly useful.

The tooth whitening compositions may comprise an opacifying agent. Any suitable opacifying agent known in the art may be used. Some examples of suitable opacifying agents include titanium dioxide, mica, mica coated titanium dioxide, polyethylene, polypropylene, polyester particulates, combinations thereof, and etc. However, the preferred opacifying agent of the present application is titanium dioxide. The level of opacifying agent is from about 0.2% to about 2.0%, preferably, from about 0.5% to about 1.5% by weight.

The process for preparing tooth whitening compositions of the present application comprises the steps of: (i) preparing a mixture comprising fluoride agent, sweetener, thickener, colorants, humectant, tartar control agent, copolymer and required amount of water; (ii) adding peroxide-polymer complex to the mixture formed in step (i), wherein said peroxide-polymer complex comprises hydrogen peroxide and vinyllactam based polymer; (iii) optionally, heating the mixture of step (ii) at a temperature of 150-160 F to result in homogenous mixture in the form of gel; (iv) adding coated abrasives to the mixture from step (iii) under vacuum and vigorous agitation to yield complete dispersion of coated abrasives; (v) breaking vacuum of step (iv), adding surfactant and detergents without any mixing; (vi) adding antibacterial and flavoring agents to step (v) and mixing under vacuum to yield complete homogenous mixture; and (vii) breaking vacuum of step (vi) and filling into the mixture tubes.

Further, certain aspects of the present invention are illustrated in detail by way of the following examples. The examples are given herein for illustration of the invention and are not intended to be limiting thereof.

### Example 1

### Tooth Whitening Composition

| Components | % in Formula |
|---|---|
| Sodium Fluoride | 0.24 |
| Sodium Saccharin | 0.30 |
| Titanium Dioxide | 1.00 |
| 70% Sorbitol Solution | 27.00 |
| Encapsulated Abrasive | 3.00 |
| Fumed Silica (Aerosil 200) | 1.50 |
| Glycerin | 10.00 |
| Carboxymethyl Cellulose (CMC | 2.00 |
| 12M31XP) | |
| Carboxymethyl Cellulose (CMC 7MXF) | 0.20 |
| Hydrogen Peroxide (Peroxydone K-30) | 6.25 |
| Sodium Lauryl Sulfate | 2.00 |
| DI Water | Q.S to100 |

### Example 2

### Stability Test

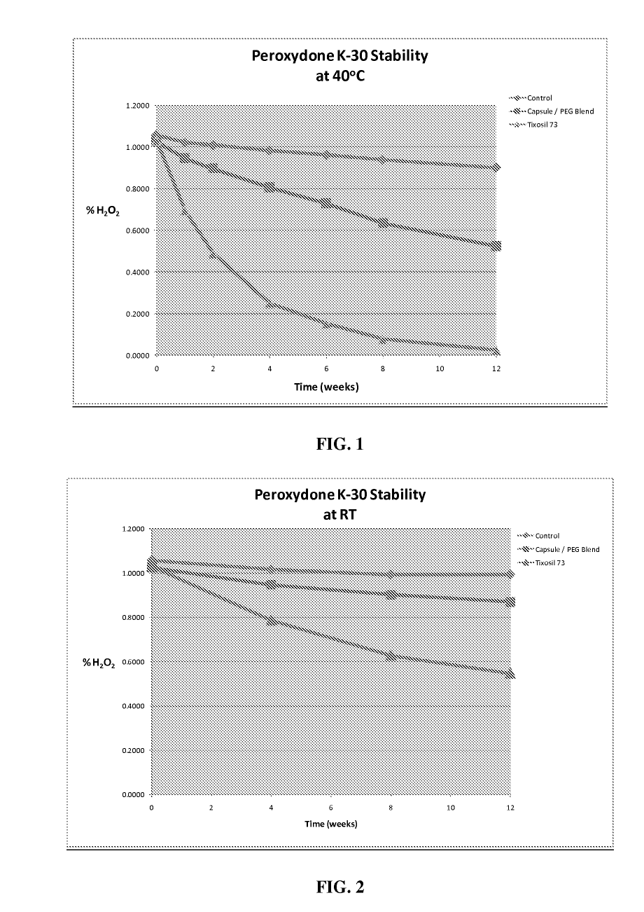

Results of a 3 month stability study are summarized in FIGS. 1 and 2. This result shows a significant improvement in the stability of Peroxydone in an aqueous system containing encapsulated Tixosil 73 vs. unencapsulated Tixosil 73. The control product containing no abrasive loses only 10% of its active oxygen over 3 months at 40° C. compared to test product containing untreated Tixosil 73 which loses all its active oxygen between 8 and 10 weeks at 40° C. Test product containing encapsulated Tixosil 73 shows a loss of about 45% of its initial peroxide after 3 months at 40° C. vs. the control product with no abrasive which undergoes a loss of 10%. However, the peroxide in the test product containing encapsulated Tixosil 73 is significantly more stable compared to the test product containing untreated Tixosil 73 by approximately 55% after 3 months at 40° C.

### Example 3

### Abrasivity Test

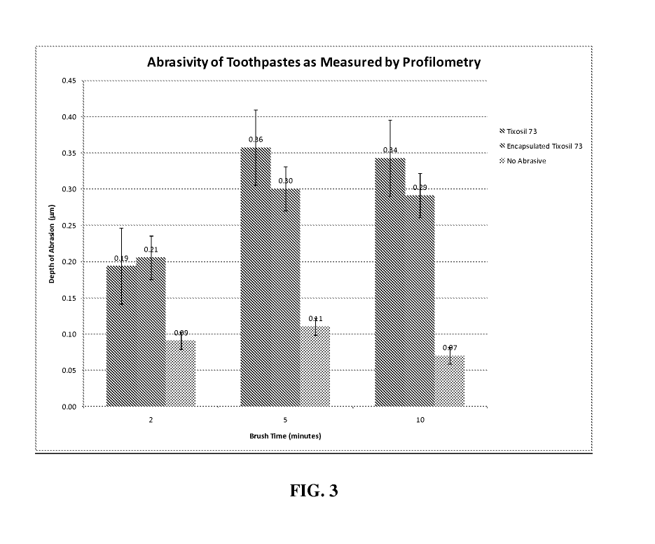

Results of the abrasivity tests are summarized in FIG. 3. After 2, 5 and 10 minute brushes there are no statistically significant differences between tests products containing untreated vs. encapsulated Tixosil 73. Also, there is very little difference in abrasivity seen between a 5 minute brush and a 10 minute brush. In all cases, the control containing no abrasive has very little effect on the acrylic panel surface and is statistically and visually less abrasive than either of the abrasive containing test products. Additionally, the performance of the abrasive containing test products is most similar after a 2 minute brush. This suggests that the encapsulated abrasive is released early in the brushing process which is important in that the average person only brushes for about 2 minutes.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

What is claimed is:

1. A water-based, peroxide-stabilized, abrasive tooth whitening composition comprising:
i. a peroxide-polymer complex comprising (a) a peroxide component or a peroxide generating component; and (b) a vinyl lactam based polymer;
ii. coated abrasive particles; and
iii. one or more orally acceptable carriers.

2. The abrasive tooth whitening composition according to claim 1, wherein said peroxide component is selected from organic peroxy compounds, peroxy acids, hydrogen peroxide, calcium peroxide, urea peroxide, sodium peroxide, magnesium peroxide, potassium peroxide, strontium peroxide, zinc peroxide, and/or carbamide peroxide.

3. The abrasive tooth whitening composition according to claim 1, wherein said vinyl lactam based component of the polymer is selected from the group consisting of N-vinyl-2-pyrrolidone, N-vinyl-2-piperidone, N-vinyl-3-methyl-pyrrolidinone, N-vinyl-3-methyl-piperidone, N-vinyl-3-methyl-caprolactam, N-vinyl-4-methyl-pyrrolidinone, N-vinyl-4-methyl-2-pyrrolidone, N-vinyl-4-methyl-piperidone, N-vinyl-4-methyl-caprolactam, N-vinyl-5-methyl-pyrrolidinone, N-vinyl-5-ethyl-2-pyrrolidone, N-vinyl-4-methyl-piperidone, N-vinyl-3-ethyl-pyrrolidinone, N-vinyl-4,5-dimethyl-pyrrolidinone, N-vinyl-5,5-dimethyl-pyrrolidinone, N-vinyl-3,3,5-trimethyl-pyrrolidinone, N-vinyl-5-methyl-5-ethyl-pyrrolidinone, N-vinyl-3,4,5-trimethyl-3-ethyl-pyrrolidinone, N-vinyl-6-methyl-2-piperidone, N-vinyl-6-ethyl-2-piperidone, N-vinyl-3,5-dimethyl-2-piperidone, N-vinyl-4,4-dimethyl-2-piperidone, N-vinyl-2-caprolactam, N-vinyl-7-methyl-caprolactam, N-vinyl-7-ethyl-caprolactam, N-vinyl-3,5-dimethyl-caprolactam, N-vinyl-4,6-dimethyl-caprolactam, N-vinyl-3,5,7-trimethyl-caprolactam, N-vinyl-2-valerolactam, N-vinyl-hexahydro-2-azepinone, N-vinyl-octahydro-2-azocinone, N-vinyl octahydro-2-azoninone, N-vinyl decahydro-2-azecinone, polyvinylpyrrolidone (PVP), poly-N-vinyl-poly-2-pyrrolidone (PVPP), poly-N-vinypoly-2-piperidone, poly-N-vinyl-poly-2-caprolactam.

4. The abrasive tooth whitening composition according to claim 1, wherein said peroxide component is hydrogen peroxide.

5. The abrasive tooth whitening composition according to claim 1, wherein said vinyllactam based polymer is polyvinylpyrrolidone or poly-N-vinyl-poly-2-pyrrolidone.

6. The abrasive tooth whitening composition according to claim 1, wherein said vinyllactam polymer is complexed with a peroxide component and said vinyllactam polymer is uncrosslinked or crosslinked.

7. The abrasive tooth whitening composition according to claim 1 comprising from about 0.1% to about 50% of said peroxide-polymer complex.

8. The abrasive tooth whitening composition according to claim 1, wherein the peroxide content of said peroxide-polymer complex is from about 1% to about 20% of said composition.

9. The abrasive tooth whitening composition according to claim 1, wherein said peroxide-polymer comprises a peroxide component and vinyllactam polymer in about a 1:1 or 1:2 molar ratio.

10. The abrasive tooth whitening composition according to claim 1, wherein said abrasive particles are selected from the group consisting of sodium metaphosphate, potassium metaphosphate, tricalcium phosphate, calcium phosphate dehydrate, anhydrous dicalcium phosphate, calcium pyrophosphate, magnesium orthophosphate, trimagnesium phosphate, calcium carbonate, baking soda, sodium hexametaphosphate, magnesium carbonate, magnesium silicate, titanium dioxide, zinc oxide, aluminum silicate, zirconium silicate, hydrated alumina, bentonite, hydrated silica, amorphous silica, silica gel or colloidal silica, alkali metal aluminosilicate complexes, silicon dioxide, alumina, and any combination thereof.

11. The abrasive tooth whitening composition according to claim 1, wherein the abrasive particles are amorphous precipitated silica.

12. The abrasive tooth whitening composition according to claim 1, wherein the coating agent for abrasive particles is selected from the group consisting of algin, alginic acid, alginate salts, camitine, carrageenan, dextrin, karaya gum, agar gum, guar gum, gellan gum, irish moss, vee gum, tara gum, okra gum, gum Arabic, acacia gum, amylopectin, pectina or pectin, ghatti gum, natto gum, tragacanth gum, xanthan gum, sclerotium gum, kelp, locust bean gum, psyllium seed, tamarind gum, destria gum, chitosan, starch, modified starch, gelatin, fish gelatin, alkyl and hydroxycellulose, microcrystalline cellulose, hydroxyethylcellulose, poly(acrylic acid), polyethylene glycol, polyvinyl alcohol, polyvinylpyrrolidone, zein, polyols, esters thereof, salts thereof and mixtures thereof.

13. The abrasive tooth whitening composition according to claim 1, wherein the coating agent for abrasive particles is fish gelatin and acacia gum.

14. The abrasive tooth whitening composition according to claim 1, wherein the coated abrasive particles are present at a level of from about 1% to about 50% of said composition.

15. The abrasive tooth whitening composition according to claim 1, wherein the abrasive particles are coated by complex coacervation.

16. The abrasive tooth whitening composition according to claim 1, wherein said orally acceptable carrier is selected from the group consisting of anticalculus agents, oral cleaning agents, bleaching or whitening agents, desensitizing agents, dental re-mineralization agents, surfactants, detergents, antibacterial agents, anticaries agents, breath freshening agents, buffering agents, pH modifying agents, sweeteners, cooling agents, humectants, bioadhesive agents, coloring agents, thickening agents, film-forming agents, oral hygiene promoting agents, herbal extracts, essential oils, wetting agents, preservatives, foaming agents, flavoring agents, medicinal ingredients, vitamins, taste masking agents, and solubilizers alone or combinations thereof.

17. The abrasive tooth whitening composition according to claim 16, wherein the re-mineralization agent is selected from calcium or fluoride ions.

18. The abrasive tooth whitening composition according to claim 16, wherein said surfactant is anionic surfactant.

19. The abrasive tooth whitening composition according to claim 16, wherein said sweetening agent is a salt of saccharin and/or sorbitol.

20. The abrasive tooth whitening composition according to claim 16, wherein said humectant is glycerine and/or sorbitol.

21. The abrasive tooth whitening composition according to claim 16, wherein said thickening agent is a cellulose derivative.

22. The abrasive tooth whitening composition according to claim 16, wherein said coloring agent is titanium dioxide.

23. The abrasive tooth whitening composition according to claim 16, wherein the composition is toothpaste.

24. The abrasive tooth whitening composition according to claim 1, wherein the pH of the composition is 4 to 6.

25. A method for tooth whitening comprising:
i. preparing the abrasive tooth whitening composition of claim 1;
ii. applying the tooth whitening composition to the teeth.

26. The tooth whitening method of claim 25, wherein the tooth whitening composition is applied to the tooth by means of flexible trays, forms, toothbrush, freehand, or fingers.

27. The tooth whitening method of claim 25, wherein the duration of contact of the tooth whitening composition with the tooth in a single session is between 2 to 60 minutes.

28. A process of preparing a water-based, peroxide-stabilized, abrasive tooth whitening composition comprising the steps of:
(i) preparing a mixture comprising fluoride agent, sweetener, thickener, colorants, humectant, tartar control agent, copolymer and required amount of water;
(ii) adding peroxide-polymer complex to the mixture formed in step (i), wherein said peroxide-polymer complex comprises hydrogen peroxide and a vinyllactam based polymer;
(iii) optionally, heating the mixture of step (ii) at a temperature of 150-160 F to result in homogenous mixture in the form of gel;
(iv) adding coated abrasives to the mixture from step (iii) under vacuum and vigorous agitation to yield complete dispersion of coated abrasives;
(v) breaking vacuum of step (iv), adding surfactant and detergents without any mixing;
(vi) adding antibacterial and flavoring agents to step (v) and mixing under vacuum to yield complete homogenous mixture; and
(vii) breaking vacuum of step (vi) and filling into tubes.
